(19) United States
(12) Patent Application Publication
(10) Pub. No.: US 2010/0041990 A1
(43) Pub. Date: Feb. 18, 2010

(54) Needle Guides for Catheter Delivery

(76) Inventors: John Schlitt, Austin, TX (US); Geoffrey S. Findlay, Austin, TX (US)

Correspondence Address: DAFFER MCDANIEL LLP, P.O. BOX 684908, AUSTIN, TX, 78768, US

(21) Appl. No.: 12/540,769

(22) Filed: Aug. 13, 2009

Related U.S. Application Data
(60) Provisional application No. 61/188,833, filed on Aug. 13, 2008.

(51) Int. Cl. A61B 8/00; A61M 19/00

(52) U.S. Cl. 600/439; 600/461

(57) ABSTRACT

Provided are needle locks and methods of using the same for ultrasound guided catheter insertion. The needle locks hereof substantially immobilizes a needle relative to an ultrasound probe to permit manipulation of a catheter or other device through the needle. The needle locks of the present invention may be used in nerve block, venous catheter insertion procedures or other invasive procedures.

### PRIORITY CLAIM

The present application claims priority to U.S. Provisional Application No. 61/188,833 filed Aug. 13, 2008 and is incorporated by reference herein.

### BACKGROUND OF THE INVENTION

1. Field of the Invention

The present invention relates generally to systems and methods for ultrasound imaging, and in particular, to systems and methods for correct placement of needles, catheters, and other medical devices using an ultrasound probe.

2. Description of Related Art

Ultrasound imaging is a medically important technique that may be used for a variety of applications including regional nerve block, line placement, tumor imaging, epidural placement, emergency room diagnosis, and catheter delivery to name a few. Present methods for catheter delivery using ultrasound guidance normally require the practitioner to hold an ultrasound probe with one hand and insert a needle with the other hand. The ultrasound probe allows the practitioner to visualize the internal organs and other sensitive structures as well as the needle, while inserting the needle into the patient.

Ultrasound guided needle insertion allows for the avoidance of critical structures which the practitioner would not want to damage or puncture during insertion of the needle. For example, care must be taken to avoid specific structures including nerves, tendons, lung and/or arteries during a regional nerve block procedure or during the placement of a central line catheter. Damage to a nerve or injection of an anesthetic solution into a nerve during a nerve block procedure can severely damage or kill the nerve. Puncturing the lung during a nerve block procedure typically requires an extended hospital stay, creating a pneumothorax and possibly requiring insertion of a chest tube. Delivery of a local anesthetic into an artery can result in patient death due to cardiac or respiratory collapse. Ultrasound visualization can provide a critical advantage for visualizing and avoiding these internal structures.

One example of the use of ultrasound visualization is the administration of regional anesthesia. During surgical procedures, it is often desirable to provide regional anesthesia on a continual basis as an infusion, particularly in the postoperative settings. A reliable means of achieving regional anesthesia is by application of a local anesthetic to a peripheral nerve or plexus of nerves. By doing so, a particular area is numbed to painful stimuli, which may occur during or after surgery, or from other forms of trauma.

A standardized medical procedure for providing regional anesthesia as a continuous infusion is the introduction of a peripheral nerve catheter adjacent to the desired nerve or nerve plexus. One common method of localizing the desired nerve or nerve plexus is by applying milliamp levels of current to an insulated needle. The nerve is then located by gradually advancing the insulated needle until the applied current induces visible muscle contraction. In order to achieve muscle contraction, however, the tip of the insulated needle must be within millimeters of the nerve. This allows only a very small margin for error on the part of the anesthesiologist, and obviously to those skilled in the art, any movement of the needle could reduce the effect of the regional anesthetic, damage the nerve, or both. Examples of this method are illustrated in U.S. Pat. Nos. 5,976,110; 7,120,487.

Another method of localizing the desired nerve or nerve plexus is by using ultrasound visualization to guide needle placement and catheter positioning. Examples of this method are illustrated in U.S. Pat. Nos. 6,743,177; 7,452,331 and U.S. Patent Application Publication Nos. 2005/0131291 and 2007/0049822. Sometimes, the nerve stimulation technique is combined with the ultrasound visualization technique. All patents and applications recited herein are incorporated by reference for all permitted purposes.

Several serious limitations exist regarding ultrasound visualization techniques for guided needle or catheter placement. First, ultrasound visualization requires that the needle be within a few millimeters of the 2-dimensional plane of visualization of the ultrasound. Further, during catheter insertion, the practitioner typically is forced to remove the ultrasound probe from the skin of the patient while holding the needle still with the other hand; then the practitioner must thread a catheter through the needle into the patient, e.g., to be placed near a nerve for a nerve block procedure or into a vein during line placement. During this portion of time it is not possible to know with any certainty where the tip of the catheter is relative to the tip of the needle. During the threading of a catheter it is typically not possible to visualize the catheter as it exits the needle as the ultrasound probe has been set down. In order to hold the ultrasound probe, hold the needle, and thread the catheter, one requires the use of a “third hand”. Since the catheter is threaded into a sterile field, this is often difficult to achieve and an assistant may not be available.

The inability of one to visualize the catheter tip during catheter placement presents a significant risk. For example, during a nerve block procedure, placement of a catheter too far through a needle could insult, damage, or kill a nerve or puncture a lung rather than remain at a location appropriate for bathing the nerve in local anesthetic. Placement of the catheter not far enough into a patient could result in an incomplete nerve block and/or fail to establish an acceptable level of anesthesia. Accidental puncturing of a blood vessel could result in central neural toxicity and/or cardiac toxicity due to, e.g., transport of an anesthetic via an artery. Failure to appropriately place a catheter accounts for a significant portion of morbidity and mortality. Clearly, there exists a need for improved methods for the placement of catheters using ultrasound guidance.

Furthermore, the desire to guide a medical device (e.g. catheter, wire, sensor, drain, tube, instrument) into a patient is not limited to anesthesia-related nerve blocks and line placement. The described method could include other anesthesia-related procedures like epidural placement. It could also include other subspecialties of medicine. Radiologists and general surgeons are commonly using ultrasound for vascular access. In these cases a wire is threaded through a needle and into a blood vessel. The procedure would be safer if the wire could be visualized as it was threaded into the body. Ultrasound is also being used commonly in the Emergency Room and Intensive Care Unit. A method of guiding other medical devices, such as a drain through a needle into a fluid collection, or guiding a chest tube through an introducer into a patient's chest, would be safer and more efficient if one were able to watch said drain or tube directly under ultrasound guidance. Surgeons could use this method to do office based ultrasound-guided biopsies as small biopsy instruments could be placed through trochars placed under ultrasound. As the portable ultrasound becomes more affordable and available the opportunity to adopt new ultrasound-guided procedures is growing rapidly.

When an anatomical structure is visualized with ultrasound, an accurate 2D image of that structure appears on the ultrasound screen. When one desires to place a medical device (needle, catheter, drain, tube, wire, sensor, etc.) near that structure, he or she must bring that foreign body under the ultrasound probe in order to visualize it. This often proves quite difficult, as the “beam” or “field of view” emitted by the probe is only a few millimeters wide. Thus, a needle guide attempts to “guide” a needle under an ultrasound probe so that it can be visualized on an ultrasound screen.

The current limitation on ultrasound needle guides is their inability to hold a needle still while pressure greater than gravity is applied. Current needle guides have the ability to guide a needle in a single axis, such as the short axis (the needle appears as a dot on the screen) or long axis (the needle appears as a line on the screen). However, when solution is injected through a needle, or when a medical device is passed into a patient through a needle, a great deal of force can be generated thereby placing the needle at risk for unwanted movement. In addition, certain medical devices meet resistance as they are threaded through a needle and into the patient's body. A significant amount of pressure must be applied to these objects that is transmitted to the needle if the needle is not fixed in place. There is currently no needle guide on the market that takes this need into account.

Another limitation of current needle guides is their inability to accommodate a variety of needle insertion sites. Most guides are small and do not extend out from the ultrasound probe. This means that the needle must be inserted within a few centimeters of the probe in order to use the guide. This is often not the optimal needle insertion site to thread a foreign body into a patient.

A final limitation of current needle guides is the inability of the user to “freehand” when necessary. On occasion, it is necessary to alter the angle of the needle in real time when performing a procedure. This might mean that the needle is not entirely in the plane of the ultrasound. It might also mean that the needle is being leaned on or “joysticked” in order to get the needle tip into the correct area of a patient. Current guides do not usually allow for this type of free movement of a needle.

### SUMMARY OF THE INVENTION

The present invention overcomes limitations in the prior art by providing improved methods and apparatuses for the placement of needles, catheters or other indwelling medical devices using ultrasound guidance. In certain aspects, the present invention provides a needle-locking device that may be attached or coupled to an ultrasound probe. The needle-locking device can allow for the substantial immobilization of a needle during an ultrasound guided needle insertion; this immobilization can allow a practitioner to insert a catheter through the needle while maintaining ultrasound visualization of the needle and catheter. In various embodiments, the tip of the catheter may be coated with a metal or other substance that allows for easy visualization of the catheter tip during catheter placement. In certain embodiments, the needle-locking device may be locked with the same hand that is used by the practitioner to hold the ultrasound probe.

Broadly speaking, the apparatus hereof operates to substantially immobilize a needle relative to an ultrasound probe during an ultrasound guided invasive procedure. The apparatus includes a support structure having a proximal region for engaging the probe and a distal region. An aperture in the distal region of the support structure is configured to receive the needle whereby one end of the needle is positionable in the ultrasound field of view. A needle lock is attached to said support structure near the aperture and selectably operable to substantially immobilize a needle disposed in the aperture.

In a broad form, a method of the present invention substantially immobilizes a needle during an ultrasound guided invasive procedure, by providing an ultrasound probe with a needle guide coupled thereto, the needle guide having an aperture for slidably receiving a needle. The ultrasound probe is manipulated over a patient's body to visualize an area of interest. A needle is slid through the aperture into the patient's body while visualizing the needle with the ultrasound probe in the area of interest. When properly positioned, the needle is locked in said aperture to substantially immobilize said needle relative to said ultrasound probe. Finally, after the needle is locked, another device, such as a catheter, is inserted through the locked needle into the area of interest.

In certain aspects, the present invention provides a needle lock attached to, including integral therewith, an ultrasound probe engagement structure, wherein the engagement structure may be coupled to an ultrasound probe. The ultrasound probe engagement structure may, in certain embodiments snap onto or slide into place on the base or side of an ultrasound probe. The needle lock may be comprised of a deformable member or other mechanical gripping structure, which can retain the needle in a fixed location relative to the needle lock, and/or an ultrasound probe that is physically or mechanically coupled to the needle lock. The needle lock may be comprised of a plastic, polymer, or rubber unit which can be brought into contact with the needle, thus immobilizing the needle relative to the needle lock and/or an ultrasound probe mechanically coupled to the needle lock. In various embodiments, the needle lock comprises a lever that may be engaged to bring the needle lock into physical contact with a needle. The needle-immobilizing unit may be detachable, disposable, and/or replaceable. In certain embodiments the needle-immobilizing unit may comprise a flexible (e.g., a polymer, rubber, etc.) unit that may be physically deformed against the needle to retain the needle in a substantially or essentially fixed position relative to the needle lock. The needle lock is preferably sterile. In certain embodiments, the needle lock is disposable.

In other aspects, the present invention provides methods for performing a nerve block and/or insertion of a central line into a vein by using a needle lock of the present invention. Furthermore, locking the needle provides a unique method to perform any procedure that would require the ability to thread a medical device into a patient under ultrasound guidance. This would be safer if it could be done under direct visualization. In various embodiments, the needle lock may be coupled to the ultrasound probe. A needle may be inserted with ultrasound guidance.

In yet further aspects, the present invention provides kits comprising a needle lock of the present invention in a container means. The kit may further comprise a catheter. The catheter may be coated with a substance which may be easily visualized via ultrasound. The substance may be a metal or a polymer. In various embodiments, the needle lock may incorporate, attach to, or be integral with a needle guide. The kit may further comprise a needle. The needle may be coated with a substance which may be easily visualized via ultrasound.

In other aspects, the present invention provides methods of teaching how to perform a nerve block or a central line placement, wherein ultrasound visualization may be continually or essentially maintained during insertion of the needle and catheter into a patient. The methods of teaching may comprise using a needle lock or needle guide comprising a needle lock of the invention.

Catheters which are contemplated by the present invention include catheters wherein a portion of one end of the catheter is coated with a ultrasound visualization agent. Said coating may be on the internal and/or external portion of the catheter. The catheter may be made via a variety of methods (e.g., dip-coating or spray coating) which would be appreciated by one of skill. In various embodiments, the catheter tip may contain a coil or piece of metal which would be easily visualized with the ultrasound machine.

A further aspect of the present invention relates to a method of teaching how to perform an ultrasound guided catheter placement or an ultrasound guided nerve block. The method may be performed by one person using continuous ultrasound monitoring of the catheter placement and/or the needle during insertion. The method may be performed using a needle guide and/or a needle lock of the present invention. In certain embodiments, the catheter tip is visualized using ultrasound monitoring during the insertion procedure. The catheter tip may be continuously or essentially continuously monitored using the insertion of the catheter.

In yet further aspects, the present invention relates to a method of demonstrating how to insert a catheter with substantially, essentially, or completely reducing movement of the needle during the insertion of the catheter, wherein a needle lock is used to stabilize the needle. In this way, the needle and/or catheter may be monitored (e.g., continuously or essentially continuously) during the insertion of the catheter. The method may be performed using a needle guide and/or a needle lock of the present invention. In certain embodiments, the catheter tip is visualized using ultrasound monitoring during the insertion procedure.

The term “coupled,” as used herein, is defined as connected, although not necessarily directly, and not necessarily mechanically. “Coupled” includes being integral therewith. The term “locked” means to substantially immobilize an object relative to another object. A break out force greater than the force of gravity would be required to move an object “locked” relative to another object.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a perspective view of an ultrasound probe insertable into an apparatus in accordance with the present invention;

FIG. 2 is a side elevational view of an apparatus of the present invention coupled to an ultrasound probe disposed in viewing relationship to a patient;

FIG. 3A is another side elevation view of another embodiment of the apparatus of the present invention;

FIG. 3B is a top view of the apparatus of FIG. 3A;

FIG. 4A is another side elevation view of another embodiment of an apparatus of the present invention;

FIG. 4B is a top view of the apparatus of FIG. 4A;

FIG. 5 is a perspective view of an ultrasound probe insertable into another embodiment of an apparatus in accordance with the present invention;

FIG. 6 is another side elevation view of another embodiment of an apparatus of the present invention;

FIG. 7A is a perspective view of an ultrasound probe coupled to an apparatus in accordance with the present invention;

FIG. 7B is a perspective view of an ultrasound probe coupled to another embodiment of an apparatus in accordance with the present invention; and

FIG. 8 is a side elevation view of another embodiment of the apparatus of the present invention coupled to an ultrasound probe.

### DETAILED DESCRIPTION OF PREFERRED EMBODIMENTS

The present invention overcomes limitations in the prior art by providing improved methods and apparatus for the placement of catheters using ultrasound guidance. In certain aspects, the present invention provides a needle locking device which may be attached or coupled to an ultrasound probe. The needle locking device can allow for the immobilization of a needle during an ultrasound guided needle insertion; this immobilization can allow a practitioner to insert a catheter or other medical device through the needle while maintaining ultrasound visualization of the needle. In various embodiments, an area near the tip of the catheter may be coated, e.g., dip coated or spray coated, with a metal or other substance, which allows for easy ultrasound visualization of the catheter tip during catheter placement. In some embodiments, a region just prior to the tip of the catheter may be coated with an ultrasound visualizing substance. Alternately, a catheter with a metal coil inserted into the catheter may be used with the present invention for visualization of the catheter during insertion. It is generally preferable that the catheters used with the present invention are sufficiently malleable or soft to reduce the possibility of the catheter puncturing or damaging an internal structure (e.g., vein, nerve, etc.) during insertion. In certain embodiments, the needle locking device may be locked using the same hand that is used by the practitioner to hold the ultrasound probe. The needle lock and apparatus of the present invention may be reversibly coupled to an ultrasound probe, which may be covered with a sterile sheath.

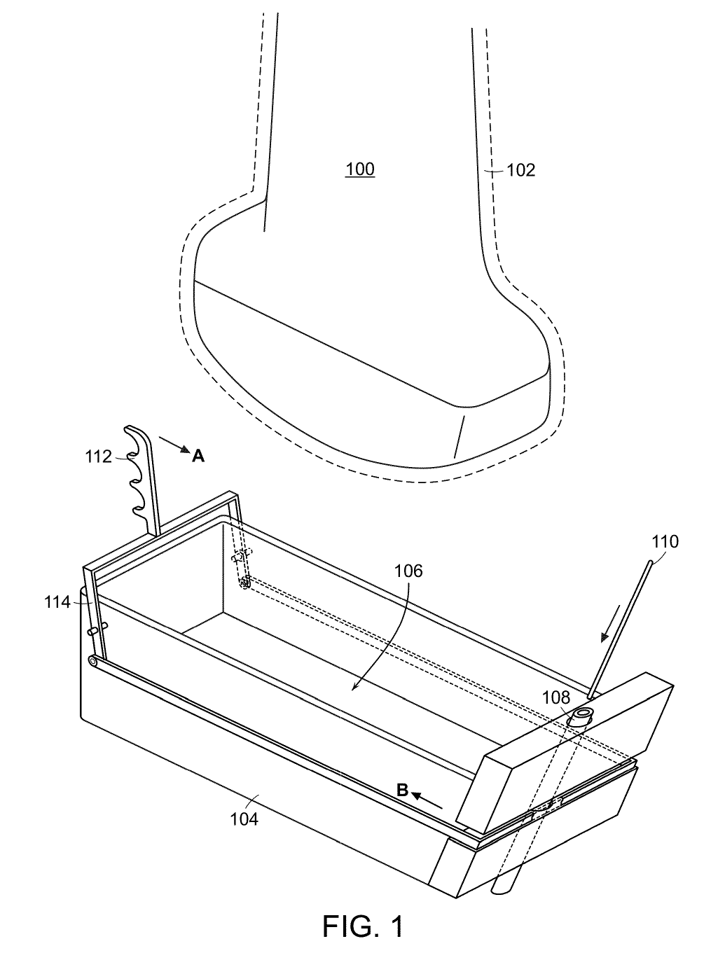

Turning to FIG. 1, an ultrasound probe 100 is covered with a protective barrier 102 in the interest of maintaining a sterile ultrasound during a procedure. The ultrasound probe 100 snaps on the support structure 104 by sliding the probe 100 into the cavity 106 of the support structure 104. The distal end of support structure 104 has an aperture 108 in the form of a deformable tube (polymeric or rubber material). The aperture 108 acts as a needle guide for receiving the needle 110 as shown. The support structure 104 includes a locking mechanism, which in the illustrated example comprises a lever 112 coupled via a linkage 114 in such a manner to contact aperture tube 108. The linkage 114 pivots as shown such that application of a force in the direction A exerts pressure in the direction B. That is, operation of the lever 112 by the practitioner in direction A deforms the aperture tube 108 locking a needle 110 disposed therein in place.

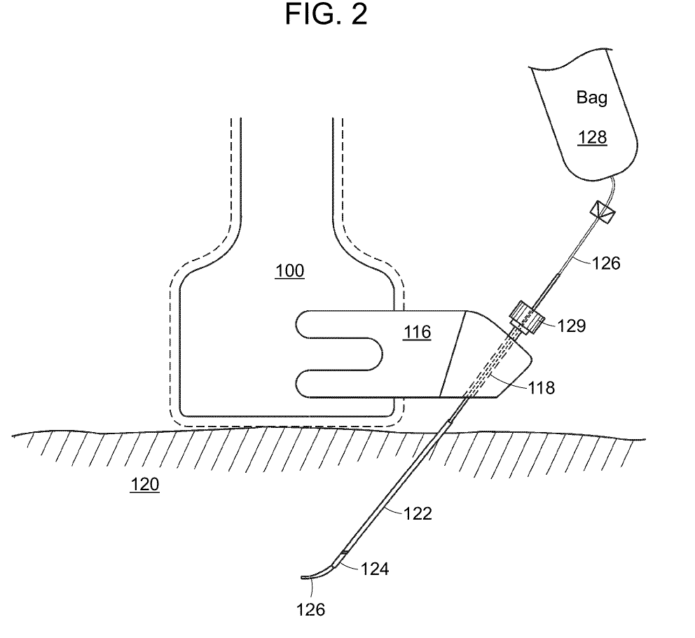

FIG. 2 shows another embodiment of the apparatus in accordance with the present invention. In FIG. 2, a C Clamp support structure 116 snaps onto the probe 100. The C Clamp 116 is specifically made and configured to engage the probe 100 in a semi-rigid fashion by simply snapping the C Clamp 116 to the probe. In the embodiment of FIG. 2, an aperture 118 extends at a fixed angle relative to the C Clamp 116. The probe 100 is place on the body of a patient 120 to visualize an area of interest in the field of view of the ultrasound prove 100.

In FIG. 2, a needle 122 has been inserted through the aperture 118 into the area or interest in the patient 120. A marker 124 can be seen which aids in identifying the position of the needle relative to the area of interest. In this FIG. 2, a catheter 126 has been inserted through the needle 122 and is disposed in the area of interest. A bag 128 containing an anesthetic agent is operatively coupled to the catheter 126 as is well known in the art.

When a needle 122 is inserted into the twist lock 129, the twist lock 129 may be twisted to essentially or completely immobilize the needle 122. Various mechanisms are available for the twist needle lock 129 to immobilize the needle 122; for example, the twist needle lock (129) may be angled to physically press against the needle 122. In other embodiments, the twist needle lock 129 may tighten a gasket or engage a stopper against the needle 122. In yet further embodiments, the twist needle lock 129 may decrease the internal diameter of the needle guide aperture 118 by wrapping one end of an elastic tube around the rotational axis of the elastic tube.

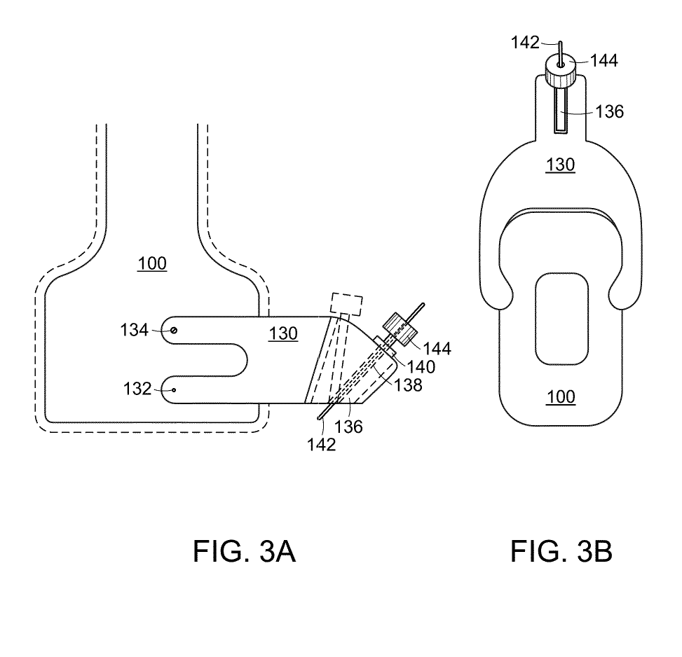

FIGS. 3A and 3B illustrate an embodiment very similar to the embodiment of FIG. 2 in that it provides a C Clamp 130 support structure which attaches to the probe 100. However, an alternative means of attachment is shown for illustrative purposes. In FIGS. 3A and 3B, the C Clamp 130 is hinged as at 132 and a set screw 134 can be tightened to lock the C Clamp 130 relative to the probe 100. Further, the aperture comprises a slot 136 receiving a rigid tube 138 to allow the positioning of the tube 138 at various angles relative to the probe 100. The tube 138 is threaded whereby set nut 140 can be tightened to lock the tube 138 in the slot 136. The needle 142 is slidably received in the tube 138 and can be locked in position by tightening the lock nut 144. A deformable gasket is housed within the lock nut 144 and upon tightening, compresses the needle 142 thereby locking the needle in place.

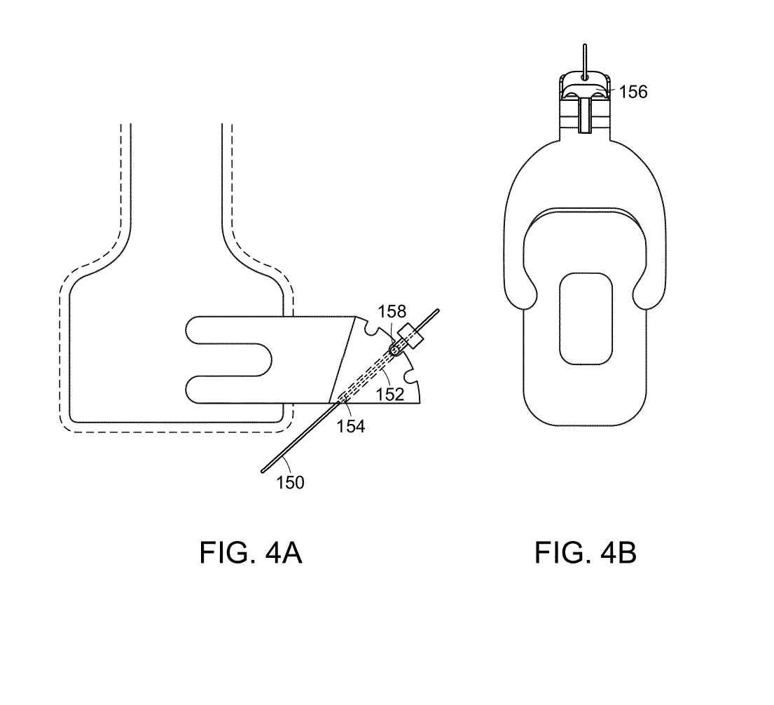

FIGS. 4A and 4B shows another embodiment similar to FIGS. 2 and 3A and 3B. In FIGS. 4A and 4B, the needle 150 is received in a tube 152, which is hinged as at 154. A spring loaded locking mechanism 156 has an arm 158 which can be moved such that the tube 152 can be locked at any of the three angles shown in FIG. 4A. The needle lock is similar to FIG. 3 in that a lock nut 160 is used.

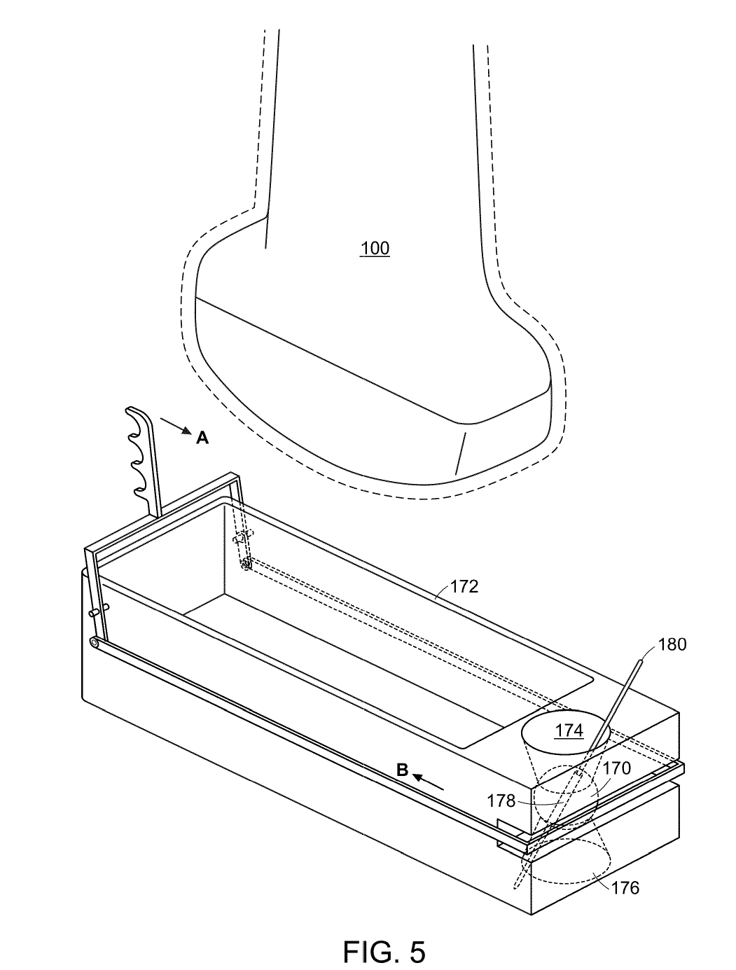

FIG. 5 depicts yet another embodiment similar to the embodiment illustrated in FIG. 1. However, in FIG. 5 a deformable ball 170 is disposed in a ball joint cavity in the support structure 172. Openings 174,176 are disposed next to the ball 170. The ball 170 includes a tubular aperture 178 for receipt of the needle 180. As can be seen, movement of the lever in direction A induces a force in direction B whereby the linkage asserts a force on the ball 170 immobilizing the ball 170 relative to the structure 172 and also compressing the ball to immobilize the needle 180 relative to the ball 170.

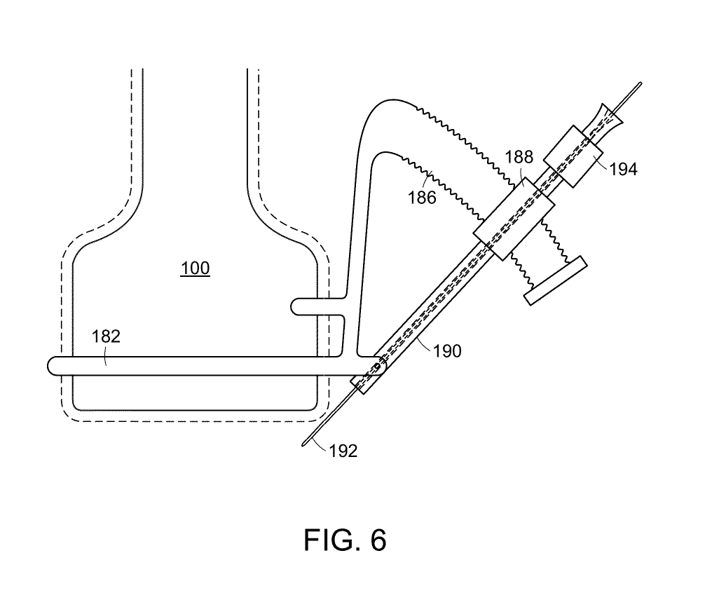

In FIG. 6 the support structure 182 slides onto the probe 100 and is additionally supported by the shoulder 184. The support structure 182 includes an elongated serrated arm 186 extending away from the probe 100 as shown. A receiver 188 is mounted to the arm 186 whereby movement of the arm 186 allows the receiver 188 to be repositioned along the serrations in the arm 186. A tubular needle guide 190 has an aperture to receive a needle 192 as shown. The needle guide 190 is hinged to the support structure 182 so that the needle 192 can be inserted into the patient at the desired angle. A lock nut 194 operates similar to the embodiment of FIGS. 2 and 3 to lock the needle 192 in place when desired.

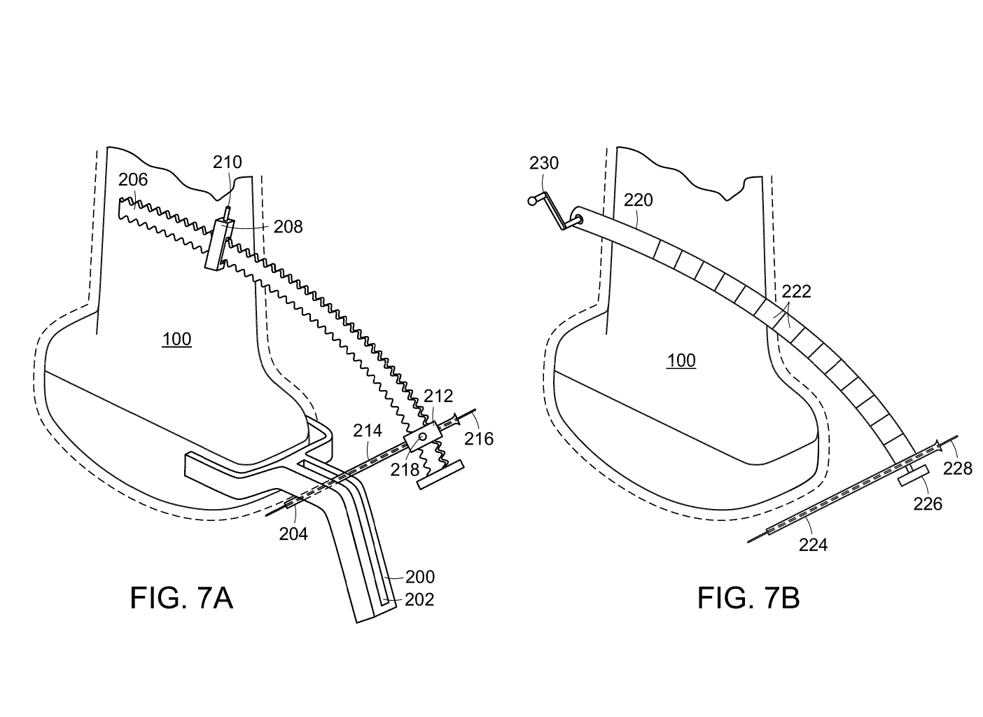

FIGS. 7A and 7B illustrate yet further embodiments of the present invention. In FIG. 7A a support structure 200 is attached to the probe 100. The support 200 has a slot 202 that acts as a needle guide to receive needle 204. The slot 202 generally limits the movement of the needle such that it must be inserted along the long axis of the field of view of the ultrasound probe 100. The serrated arm 206 extends through a slot in the probe attachment point 208 and can be locked in place by operation of the button 210. The receiver 212 operates similar to the embodiment of FIG. 6 to allow repositioning of the receiver 212 relative to the arm 206. A needle guide 214 extends from the receiver 212 through the slot 202 and as can be appreciated, allows for many positioning possibilities of the needle guide 214 relative to a patient. A needle 216 is received in the needle guide 214 and can be locked in place by operation of the set screw 218.

FIG. 7B is a variation of FIG. 7A but eliminates the support structure and slot. Thus, while FIG. 7A allows for many positioning possibilities, the embodiment of FIG. 7B is almost a “freehand” in that the needle guide can be positioned almost anywhere relative to a patient. In FIG. 7B the arm 220 is a series of segmented, interconnected tapered tubes 222. The needle guide 224 is rigidly attached to the arm 220. A needle lock takes the form of a set screw 226 for locking a needle 228 in the needle guide 224. The proximal end of the arm 220 includes a lever 230 which is connected to a semi-elastic wire (not shown) internal to the arm 220. In this manner operation of the lever 230 loosens the wire allowing repositioning of the segmented tubes 222 relative to each other, including the angle of the arm 220. Using the lever 230 to tighten the wire locks the segmented tubes 222 relative to each other. In the embodiment of FIG. 7B the arm 220 is not locked to the probe 100 by a mechanical mechanism. Instead, during use the practitioner grasps the probe 100 with one hand (e.g. right hand) and with the fingers of the same hand grasps the arm 220. Thus, with one hand the practitioner controls the position of the probe 100 and the needle guide 224 relative to the patient. When the needle 228 is positioned in the area of interest in the patient, the practitioner locks the needle 228 in place by operating the set screw 226.

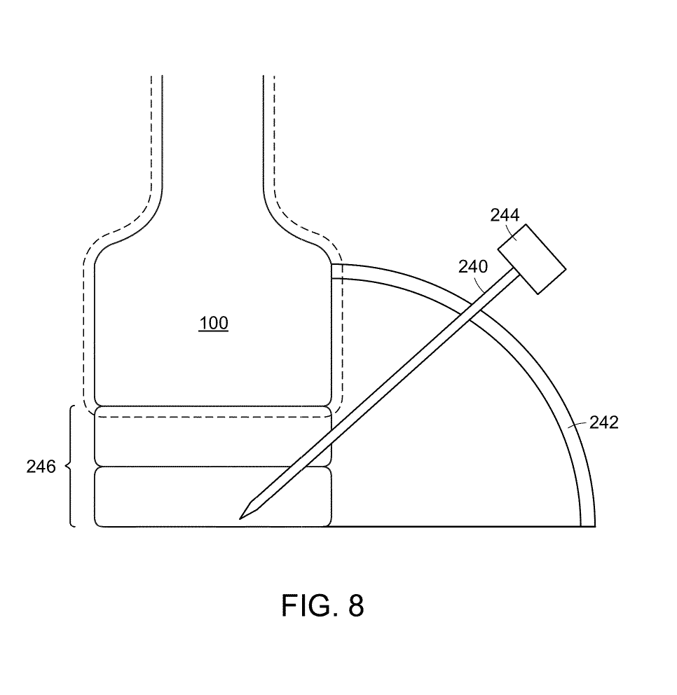

FIG. 8 illustrates another embodiment for locking a needle 240 relative to a probe 100. In FIG. 8, a slotted support structure 242 similar to FIG. 7A is used, along with a lock nut 244 similar to the lock nut of FIG. 3A. In FIG. 8, a gel cake structure 246 is affixed to the probe 100. Such a gel cake 246 is translucent to the ultrasound, yet acts as a penetrable sponge to allow insertion of the needle 240. Of course, care must be taken to not contaminate the end of the needle with gel cake material or to evacuate such material prior to inserting a catheter. In any event, the density of the gel cake 246 is such that it will hold the needle 240 in place—at least against gravity—once the needle is inserted. The practitioner can overpower this frictional force to withdraw the needle 240 from the gel cake 246 if necessary.

Although the figures display needle guides that allow for insertion of the needle in the same plane as ultrasound probe scanning field (“long axis”), in certain embodiments, it may be desirable to allow for insertion of the needle perpendicular to the ultrasound probe scanning field (“short axis”). Thus, while in certain embodiments it may be desirable to be able to visualize the needle during in parallel with the long axis of the field of view of the ultrasound probe, in other embodiments, the needle guides and needle locks of the present invention may a configured to allow for insertion of the needle perpendicular to the ultrasound scanning field (“short axis”). For example, as would be appreciated by one of skill, various radiology procedures utilize insertion of a needle, e.g., for performing a biopsy, using ultrasound guidance while inserting the needle in the short axis. Thus the support structures of the present invention may be configured to reversibly couple (e.g., snap onto) an ultrasound probe to allow for needle insertion in the either the short axis or the long axis.

The apparatus and methods of the present invention address deficiencies associated with current needle guides. First, the apparatus locks the needle against the ultrasound probe allowing for significant pressure to be placed on the needle. This allows the user to let go of the needle with one hand, thus freeing that hand up to thread a catheter or inject a solution or manipulate another medical device. When the catheter is threaded or solution injected, the needle does not move and remains visible in the ultrasound plane. Thus, the user can directly visualize catheter placement and/or injection without the need for a “third hand.” Next, the apparatus and needle lock allows the user a much larger variety of needle angles and needle insertion sites. In certain depictions, a needle guide can even accommodate negative needle angles, such as illustrated in FIGS. 7A and 7B, for instances when the needle might need to travel up to meet the ultrasound probe. This often allows one to align a needle in an optimal angle to thread a medical device at an appropriate angle into a patient. Lastly, certain versions of the apparatus and needle lock do not contain a needle guide feature at all, such as illustrated in FIG. 7B. This allows the user to introduce the needle at any possible angle in any possible direction while still locking the needle in place for medical device insertion. This would often be desirable for ultrasound experts that find the limitations of a needle guide cumbersome.

A typical scenario for the practice of the method hereof is illustrated with reference to FIG. 2 and involves a nerve block procedure where it is desirable to inject local anesthetic solution around a nerve structure to render an extremity insensate. This allows patients undergoing major orthopedic procedures to have little to no pain afterwards. In this scenario, the ultrasound probe 100 is used to visualize the nerve prior to injection. Once the nerve is visualized, a needle 122 is placed near the nerve under ultrasound guidance and local anesthetic is then injected around the nerve. Next, a plastic catheter 126 is then threaded through the needle 122, next to the nerve, so that medication can continue to be dripped onto the nerve from bag 128 and pain can be controlled for days after surgery. Currently, after local anesthetic in injected, the operator must remove the ultrasound probe 100 from the patient in order the thread the catheter 126. This is because one hand is needed to hold the needle 122 in place while the other hand is needed to apply pressure to thread the catheter 126.

Using method of the present invention, the operator would lock the needle 122 relative to the ultrasound probe 100 by tightening the lock nut 129, allowing one hand to use the ultrasound probe 100 and stabilize the needle 122 to accommodate catheter threading. The other hand could then thread the catheter 126. In this scenario the ultrasound probe 100 remains on the patient throughout allowing the operator to see the catheter 126 exit the needle tip and move into the area of interest near the nerve structure. If the catheter 126 did not travel along its intended route the operator would immediately recognize this in the ultrasound image and redirect the catheter tip—the marker 124 aids this visualization. This would not only increase success of this procedure, but help avoid these catheters getting into dangerous structures where they are not wanted.

In a variant of such a nerve block procedure, the user might choose to use the needle lock alone as shown in FIG. 7B and “freehand” the needle. The user might choose to use the needle lock as offered in a needle lock/needle guide combination. The user may also choose to use the needle lock as part of an ultrasound guided catheter tray or kit that contains a support structure, needle lock, needle guide, needle, catheter, and other necessary items to perform the procedure. In all of the above-mentioned examples the operator would need the needle lock in order to thread a catheter under direct ultrasonographic visualization without assistance from an additional operator.

A. Performing a Peripheral Nerve Block and Threading a Peripheral Nerve Catheter Using the Surethread Needle-Locking System

The below method is provided as an example of a method for performing a peripheral nerve block using an apparatus of the present invention. One of skill will recognize that various modifications may be made to the below technique. First, the operator will prep and drape the patient in a sterile fashion. Next the operator will open the catheter kit and put on sterile gloves. The ultrasound probe will then be prepared with a sterile sheath and the needle-locking snap-on guide. Sterile ultrasound gel will be placed over the site to be blocked, and the probe will be placed on the skin. The nerve to be blocked will be optimized with the ultrasound. Once the image is optimized, the block needle will be placed, in long axis, under direct visualization, in proximity to the nerve. Local anesthetic will then be injected and surround the nerve. Once this has been completed, the needle will be “locked” in place with the needle locking mechanism.

Once the needle is fixed, the operator will release the needle, while maintaining control of the ultrasound probe. The block needle is locked in place by the ultrasound probe and needle-locking system. The operator will then thread the peripheral nerve catheter through the needle and advance it until it reaches the needle tip. At this point, the operator will continue to advance the catheter and carefully watch the catheter tip as it exits the needle and travels towards the nerve in question. This can now be performed in real-time where previously the operator would have to put the ultrasound probe down while anchoring the needle with one hand and threading the catheter with the other.

Once the catheter tip is confirmed in the proper location, the needle can be unlocked while it is backed out over the catheter. The catheter can then be secured in place and is ready to use.

B. Performing a Central Venous Access Using the Surethread Needle Locking System

Needle locks of the present invention may also be used to perform a central venous access. Again, one of skill will recognize that various modifications may be made to the following methods. First, the operator will prep and drape the patient in sterile fashion. Next the operator will open the central line kit and put on sterile gloves. The ultrasound probe will then be prepared with a sterile sheath and the needle-locking snap-on guide. Sterile ultrasound gel will be placed over the vascular structures and the probe will be placed on the skin. The vessels will be imaged in short axis then the probe will be turned to isolate the vascular structure in question in the long axis. Once the image is optimized, the access needle will be placed in long axis, under direct visualization, into the vascular structure. Once this has been completed, the needle will be “locked” in place with the needle locking mechanism.

Once the needle is fixed, the operator will release the needle, while maintaining control of the ultrasound probe. The access needle will now be locked in place by the ultrasound probe and needle-locking system. The operator will then thread a wire through the needle and carefully watch the wire as it threads into the vessel in question. This can now be performed in real-time where previously the operator would have to put the ultrasound probe down while anchoring the needle with one hand and threading the wire with the other.

Once the wire has been confirmed in the correct vessel the needle can be removed, the Seldinger technique can be used, the vessel dilated and a central venous catheter threaded into place.

All of the methods and apparatuses disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the method described herein without departing from the concept, spirit and scope of the invention. More specifically, it will be apparent that certain features from one embodiment may be substituted for other features described herein, or in some cases omitted, while the same or similar results would be achieved. The steps in a method or procedure can be in any order unless otherwise indicated. All such similar substitutes and modifications apparent to those skilled in the art are deemed to be within the spirit, scope and concept of the invention as defined by the appended claims.

What is claimed is:

1. An apparatus for substantially immobilizing a needle relative to an ultrasound probe during an ultrasound guided invasive procedure, comprising:
a support structure having a proximal region for engaging the probe and a distal region;
an aperture in said distal region configured to receive said needle whereby one end of said needle is positionable in the ultrasound field of view; and
a needle lock attached to said support structure near said aperture and selectably operable to substantially immobilize a needle disposed in said aperture.

2. The apparatus according to claim 1, wherein said ultrasound probe has a field of view, and said aperture comprises a slot operable to permit manipulation of said needle in said field of view.

3. The apparatus according to claim 1, wherein said support structure comprises a cradle for slidably engaging said ultrasound probe.

4. The apparatus according to claim 1, wherein said support structure includes a C-clamp for engaging said probe.

5. The apparatus according to claim 1, wherein said support structure is adapted for hinged relationship to said probe, permitting selectable orientation of said aperture relative to said probe.

6. The apparatus according to claim 1, wherein said needle lock comprises a deformable member including at least a portion of said aperture, operable to lock said needle in said aperture when a force is applied to said deformable member.

7. The apparatus according to claim 6, wherein said deformable member comprises a compressible ball coupled to said support structure.

8. The apparatus according to claim 1, wherein said support structure distal region includes an arm with said needle lock attached thereto.

9. The apparatus according to claim 8, wherein said needle lock is extensible relative to said probe.

10. The apparatus according to claim 1, wherein said support structure comprises a permeable cake attachable to said probe, and configured for receiving a needle inserted therein.

11. A method for substantially immobilizing a needle during an ultrasound guided invasive procedure, comprising:
providing an ultrasound probe with a needle guide coupled thereto, the needle guide having an aperture for slidably receiving a needle;
manipulating the ultrasound probe over a patient's body to visualize an area of interest;
sliding a needle through said aperture into the patient's body;
visualizing the needle with the ultrasound probe in the area of interest;
locking the needle in said aperture to substantially immobilize said needle relative to said ultrasound probe; and
after said needle is locked, inserting an other device through said needle into said area of interest.

12. The method according to claim 11, further comprising a nerve block procedure, and wherein said other device comprises a catheter adaptable for receiving anesthesia.

13. The method according to claim 11, further comprising a venous catheter placement, and wherein said other device is a venous catheter.

14. The method according to claim 11, further comprising an extraction procedure.

15. A system for substantially immobilizing a needle relative to an ultrasound probe, comprising:
a support structure having an aperture for slidably receiving a needle;
an elongated needle adapted for sliding engagement in said aperture;
a catheter adapted for slidable receipt within said needle, and having a lumen operable for transmission of fluids therein; and
said support structure including a lock for selectably immobilizing said needle relative to said ultrasound probe.

16. The system according to claim 15, wherein said needle includes a marker disposed near a puncture end to aid in visualization of the needle with said probe.

17. The system according to claim 15, wherein said support structure includes an arm attached to said lock whereby the position of said lock relative to said probe can be fixed.

18. The system according to claim 15, wherein said lock comprises a deformable member with said aperture disposed therein.

19. The system according to claim 18, wherein said deformable member comprises a compressible gasket, and said lock includes a compression nut.
